(19) United States
(12) Patent Application Publication
(10) Pub. No.: US 2024/0139240 A1
(43) Pub. Date: May 2, 2024

(54) SMALL EXTRACELLULAR VESICLES FOR ATTENUATING POST-OPERATIVE PAIN

(71) Applicant: DREXEL UNIVERSITY, Philadelphia, PA (US)

(72) Inventor: Seena K. Ajit, West Windsor, NJ (US)

(21) Appl. No.: 18/384,625

(22) Filed: Oct. 27, 2023

Related U.S. Application Data
(60) Provisional application No. 63/420,435, filed on Oct. 28, 2022.

(51) Int. Cl. A61K 35/15; A61K 9/00; A61K 45/06; A61P 25/04; C12N 5/0786

(52) U.S. Cl. CPC A61K 35/15; A61K 9/0019; A61K 45/06; A61P 25/04; C12N 5/0645

(57) ABSTRACT

Described herein is a method of treating, ameliorating, and/or preventing post-operative pain in a subject using certain compositions recited in the disclosure. The method comprises administering to the subject a therapeutically effective of a composition comprising a small extracellular vesicle (sEV) or an exosome released by a macrophage. Also described herein is a method of performing surgical operation. The method comprises performing a surgery on the subject, as well as administering to the subject the composition comprising the sEV or exosome herein.

### CROSS-REFERENCE TO RELATED APPLICATIONS

The present application claims priority under 35 U.S.C. § 119(e) to U.S. Provisional Patent Application No. 63/420,435, filed Oct. 28, 2022, which is incorporated herein by reference in its entirety.

### STATEMENT REGARDING FEDERALLY SPONSORED RESEARCH

This invention was made with government support under 5R01NS102836-05 awarded by National Institutes of Health. The government has certain rights in the invention.

### BACKGROUND

Most patients undergo surgical procedures experience postoperative pain. Postoperative pain is a type of acute pain in response to the surgical trauma, and is characterized in inflammatory reactions and afferent neuronal barrages. Postoperative pain is not limited to the site of surgery. Rather, patients can feel pain in areas distal from the site of surgery, such as muscle pain, throat pain and movement pain.

Conventionally, postoperative pain is managed by, among others, opioids, nonsteroidal anti-inflammatory drugs (NSAIDs), acetaminophen, and ketamine. The current methods of postoperative pain management are not ideal. The effectiveness of NSAIDs and acetaminophen are limited, and NSAIDs have been reported to contribute to the chronicification of pain and delay wound healing (see e.g., Parisien et al., Science Translational Medicine 14, 2022). The administration of opioids and ketamine can potentially lead to the dependence, especially in patients who are already exposed to these substances.

Accordingly, there is a need for novel methods for managing postoperative pain. The present invention addresses this need.

### SUMMARY

In some aspects, the present invention is directed to the following non-limiting embodiments:

Method of Treating, Ameliorating, and/or Preventing Post-Operative Pain

In some aspects, the present invention is directed to a method of treating, ameliorating, and/or preventing post-operative pain in a subject.

In some embodiments, the method comprises administering to the subject a therapeutically effective of a composition comprising a small extracellular vesicle (sEV) or an exosome released by a macrophage.

In some embodiments, the sEV or exosome is derived from the macrophage using lipopolysaccharide (LPS) stimulation.

In some embodiments, the sEV or exosome is derived from the macrophage without using lipopolysaccharide (LPS) stimulation.

In some embodiments, the composition is administered to the subject before, during or after the operation takes place.

In some embodiments, the composition is administered to the subject at least 1 day, 2 days, 3 days, 4 days, 5 days, 6 days, 7 days, 2 weeks, or more than 2 weeks before the operation takes place.

In some embodiments, the composition is administered at least 1 day, 2 days, 3 days, 4 days, 5 days, 6 days, 7 days, 2 weeks, or more than 2 weeks after the operation takes place.

In some embodiments, the subject is further administered at least one additional analgesic drug.

In some embodiments, the amount of the at least one additional analgesic drug administered to the subject along with the composition is lower than the amount of the at least one analgesic drug that needs to be administered to the subject in the absence of the composition to provide a similar level of post-operative pain relief,

In some embodiments, the duration of the pain relief provided by the amount of the at least one additional analgesic drug administered to the subject along with the composition is longer than the duration of the pain relief provided by the same amount of at least one additional analgesic drug administered in the absence of the composition.

In some embodiments, the macrophage is allogenic with respect to the subject, or the macrophage is syngenic with respect to the subject.

In some embodiments, the macrophage is obtained from the subject.

In some embodiments, the macrophage is obtained from a donated blood.

In some embodiments, the sEV or exosome is administered to the subject parentally.

In some embodiments, the subject is a human.

### Method of Performing a Surgical Operation

In some aspects, the present invention is directed to a method of performing a surgical operation on a subject.

In some embodiments, the method comprises: performing a surgery on the subject; and administering to the subject an effective amount of a composition comprising a small extracellular vesicle (sEV) or an exosome released by a macrophage.

In some embodiments, the sEV or exosome reduces a post-operative pain caused by the surgery.

In some embodiments, the sEV or exosome is derived from the macrophage using lipopolysaccharide (LPS) stimulation.

In some embodiments, the sEV or exosome is derived from the macrophage without using lipopolysaccharide (LPS) stimulation.

In some embodiments, the composition is administered to the subject before, during or after the surgery takes place.

In some embodiments, the composition is administered to the subject at least 1 day, 2 days, 3 days, 4 days, 5 days, 6 days, 7 days, 2 weeks, or more than 2 weeks before the surgery takes place.

In some embodiments, the composition is administered at least 1 day, 2 days, 3 days, 4 days, 5 days, 6 days, 7 days, 2 weeks, or more than 2 weeks after the surgery takes place.

In some embodiments, the method further comprises administering to the subject at least one additional analgesic drug.

In some embodiments, the amount of the at least one additional analgesic drug administered to the subject along with the composition is lower than the amount of the at least one analgesic drug that needs to be administered to the subject in the absence of the composition to provide a similar level of post-operative pain relief.

In some embodiments, the duration of the pain relief provided by the amount of the at least one additional analgesic drug administered to the subject along with the composition is longer than the duration of the pain relief provided by the same amount of at least one additional analgesic drug administered in the absence of the composition.

In some embodiments, the macrophage is allogenic with respect to the subject.

In some embodiments, the macrophage is syngenic with respect to the subject.

In some embodiments, the macrophage is obtained from the subject.

In some embodiments, the macrophage is obtained from a donated blood.

In some embodiments, the sEV or exosome is administered to the subject parentally.

In some embodiments, the subject is a human.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIG. 1 illustrates certain aspects of the preparation of small extracellular vesicles (sEVs) preparation process, in accordance with some embodiments.

FIG. 2 illustrates certain aspects of the behavioral methods used to assay subjects' mechanical pain thresholds (von Frey test) and thermal pain thresholds (Hargreaves test), in accordance with some embodiments.

FIGS. 3A-3B: confirmation of mechanical and thermal hypersensitivity following incision in mouse model of post operative pain. FIG. 3A Paw withdrawal threshold in 8-week-old male C57BL/6 mice that underwent paw incisional surgery. FIG. 3B: Paw withdrawal latency to a radiant heat source using the Hargreaves assay. Base line values and 5 days post-surgery shown (n=6).

FIGS. 4A-4C demonstrate that therapeutic intraplantar injections of sEVs (Exo+/− attenuate pain hypersensitivity in a dose dependent manner in the paw incisional model, in accordance with some embodiments. Eight-week-old male C57BL/6 mice underwent paw incisional surgery followed by an intraplantar injection of PBS, or 5/10 μg Exo(−) or Exo(+). Mechanical sensitivity was determined using von Frey filaments and thermal sensitivity by Hargreaves method in postoperative sEV or PBS treated mice. Statistical analysis was determined by multiple comparisons two-way ANOVA *p<0.05, **p<0.01, ****p<0.0001, n=5.

FIGS. 5A-5C demonstrate that therapeutic intrathecal injections of sEVs (Exo+/) attenuate pain in a dose dependent manner in the paw incisional model. Eight-week-old male C57BL/6 mice underwent paw incisional surgery followed by an intrathecal injection of PBS, or 5/10 μg Exo(−) or Exo(+), 5 mice to each group. Mechanical sensitivity was determined using von Frey filaments and thermal sensitivity by Hargreaves method in postoperative or PBS treated mice. Statistical analysis was determined by multiple comparisons two-way ANOVA *p<0.05, **p<0.01, ****p<0.0001, n=5.

FIGS. 6A-6C demonstrate that prophylactic intrathecal injections of sEVs (Exo+/−) attenuate pain hypersensitivity in the paw incision model of postoperative pain, in accordance with some embodiments. Eight-week-old male C57BL/6 mice got intrathecal injections of PBS or 10 μg of Exo(+) or Exo(−). Two weeks later, they all underwent paw incisional surgery. Mechanical sensitivity was determined using von Frey filaments and thermal sensitivity by Hargreaves method in postoperative sEV or PBS treated mice. Statistical analysis was determined by multiple comparisons two-way ANOVA *p<0.05, **p<0.01, ****p<0.0001, n=5.

FIGS. 7A-7C: effect of prophylactic intraplantar injections of sEVs (Exo+/−) on pain hypersensitivity in the paw incision model of postoperative pain, in accordance with some embodiments. Eight-week-old male C57BL/6 mice got intraplantar injections of PBS or 10 μg of Exo(+)/Exo(−). Two days later, they all underwent paw incisional surgery. Mechanical sensitivity was determined using von Frey filaments and thermal sensitivity by Hargreaves method in postoperative sEV or PBS treated mice. Statistical analysis was determined by multiple comparisons two-way ANOVA *p<0.05, **p<0.01, ****p<0.0001, n=5.

FIGS. 8A-8B demonstrate that intraplantar inflammatory agent induces acute macrophage infiltration and shifts in phenotypic markers, in accordance with some embodiments. Eight-week-old male C57BL/6 mice underwent intraplantar CFA injections and were sacrificed 7 days post injection. Ipsilateral or contralateral paw was collected, and skin and muscle were digested into a single cell suspension using dispase, collagenase & hyaluronidase. Cells were blocked, live dead and surface stained and analyzed by flow cytometry. Cells are represented as absolute # or % of CD45 cells after gating on all live CD45+ cells. Ipsilateral paw shows increase in CD11b+F4/80+ and CD11b+F4/80− cells, while a decrease in CD11b-F4/80+ cells. (n=1).

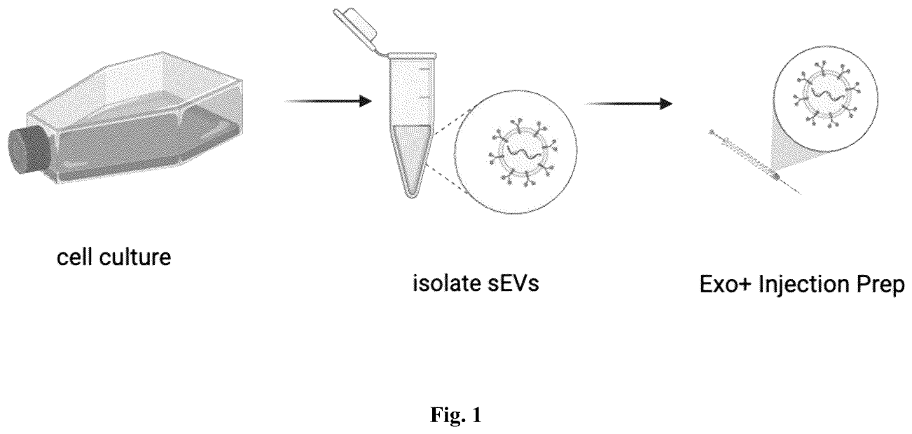

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

In certain embodiments, the present disclosure relates to the use of small extracellular vesicles (sEVs) or exosomes released by macrophages in attenuating post-operative pain. Exosomes are 30-150 nm small sEVs that carry mRNAs, miRNAs, proteins, and/or lipid mediators to recipient cells via circulation and play a key role in intercellular communication. The composition of cargo molecules differs depending on the cells releasing them, and sEVs can profoundly modulate the properties of target or recipient cells upon uptake.

sEVs have been shown to attenuate inflammatory pain (Jean-Toussaint et al., Brain Behav Immun. 2021 May; 94: 210-224).

sEVs confer therapeutic benefits by attenuating or stimulating an immune response. In complete Freud adjuvant (CFA) mouse model of inflammatory pain showed that sEVs from RAW 264.7 macrophage cells attenuate mechanical allodynia (1 μg, administered intrathecally (i.t.)) or thermal hypersensitivity (0.5 μg, administered in paw) in C57BL/6J mice. I.t. administration of sEVs derived from RAW 264.7 cells without or with lipopolysaccharide (LPS) stimulation (referred to as Exo− and Exo+ respectively) two weeks prior to CFA administration, produced a prophylactic effect in CFA model3.

Since normal pain perception is a protective feature that is crucial for survival, the present study also examined prophylactically treated mice for baseline pain perception and inflammation. The present studies showed that sEVs from RAW 264.7 macrophage cells did not induce inflammation or alter basal pain threshold in naïve or PBS treated mice, indicating that sEVs by themselves do not cause any adverse responses. Thus, sEVs have the potential to prophylactically treat pain while preserving homeostatic pain perception. This provides a paradigmatic shift in development of pain therapies. In certain embodiments, sEVs can benefit soldiers in combat and workers at high risk for occupational injuries as well as minimize post-surgical chronic pain and decrease opioid prescriptions.

However, whether sEVs are effective in attenuating pains caused by physical wounds in response to, e.g., incisions, such as post-operative pain, is far from certain. This is due to the fact that, while post-operative pain and inflammatory pain share some similarities, there exists a number of significant differences between the nature of post-operative pain and inflammatory pain, including those listed in Table 1 below:

TABLE 1
Differences between post-operative pain and inflammatory pain

| | Post-operative pain (POP) | Inflammatory pain (IP) |
|---|---|---|
| Causes | POP arises directly from surgical | IP can occur without surgical trauma, |
| | trauma. In a surgical operation, | such as in conditions like arthritis. |
| | tissues are incised, and often | |
| | manipulated, leading to nociceptive | |
| | pain. | |
| Nociceptive vs. | POP is primarily nociceptive in | IP can have both nociceptive and |
| Neuropathic | nature, resulting from tissue damage | neuropathic components. Inflammation |
| Components | and the activation of nociceptors. | can sensitize neurons leading to |
| | | neuropathic features like hyperalgesia |
| | | and allodynia. |
| Temporal | POP is typically acute and immediate, | IP can be acute or chronic, depending |
| Profile | with the intensity gradually decreasing | on the underlying condition. Chronic |
| | as the healing process progresses. | inflammatory pain conditions often |
| | | involve persistent immune responses. |
| Tissue | POP involves not only the immediate | IP originates from the body's response |
| Repair and | trauma but also the subsequent | to pathogens, damaged cells, or |
| Inflammation | healing and repair processes, which | autoimmune reactions, leading to |
| | can contribute to ongoing pain. | inflammation. IP may persist even |
| | | after inflammation has subsided. |
| Response to | POP typically responds well to | IP may require a broader range of |
| Analgesics | opioids and other analgesics that | analgesics, including anti-inflammatory |
| | target nociceptive pathways. | drugs, corticosteroids, and drugs |
| | | targeting neuropathic components. |
| Predictability | The onset and intensity of POP are | Onset and intensity of IP can be more |
| | often predictable based on the type | variable, depending on the course of |
| | and extent of the surgery. | the underlying inflammatory condition. |

To test whether sEVs are effective in attenuating pains caused by physical wounds or incisions, such as post-operative pain, the present study developed an animal model of post-operative pain, and administered sEVs to the test animals both before and after the introduction of incisions.

The data presented herein show that macrophage-derived sEVs can attenuate post-operative pain therapeutically (administered immediately after surgery). In certain embodiments, prophylactic administration of sEVs (administered 2 weeks prior to surgery) decreases the dose and duration of analgesic needed for pain relief, and is efficacious in both syngeneic and allogenic mouse models of post-operative pain.

In some embodiments, pre-emptive analgesia defined as a pre-operative treatment, is more effective than the identical treatment administered after the incision. The concept of multimodal analgesia advocates combined use of different classes of pharmacological agents for reducing acute post-operative pain while limiting perioperative opioid consumption and opioid-related adverse events. Use of sEVs can address both these concepts directly or indirectly via the anti-inflammatory miRNA cargo in RAW 264.7 derived sEVs and immune modulation. Cell free vaccines utilizing sEVs enable novel immunotherapies that overcome some of the challenges associated with the use of cells (stem cells) in clinical settings. For example, sEVs are amenable to regulated manufacturing process and long-term storage, and eliminate risks associated with in vivo cellular replication and lodging of cells in microvasculature. Preclinical studies using sEVs from allogenic and syngeneic sources can provide insights on safety and efficacy of sEVs as a pain therapeutic. Preliminary studies showed that sEVs from macrophage did not alter basal pain threshold indicating that injecting sEVs did not have a negative impact in pain perception, but attenuated pain induced by inflammatory agents. sEVs act as natural, non-toxic membranous nano-carriers of bio-macromolecules for effective delivery to injured area. Novel strategies to attenuate acute post-operative pain will benefit those undergoing surgery.

The therapeutic and prophylactic efficacy of sEVs can be evaluated in a mouse model of post-operative pain. The synergistic role of sEVs is assessed to determine whether sEVs can a) lower the efficacious dose of commonly prescribed analgesic drugs and/or b) increase the duration of pain relief in this model. Comparative (sEVs vs drugs) and combinatorial analysis (sEVs and drugs) with morphine and celecoxib can be performed. The studies can determine if sEVs can be a novel and safe treatment option for post-operative pain. The present study also studies the therapeutic efficacy of sEVs derived from allogenic and syngeneic macrophages in post-operative pain model.

Accordingly, in some aspect, the present invention is directed to a method of reducing a pain in response to a physical wound, such as a method of treating, ameliorating, and/or preventing post-operative pain in a subject.

In some aspects, the present invention is directed to a method of performing a surgical operation in a manner that reduces the post-operative pain.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

Unless defined otherwise, all technical and scientific terms used herein have the same meaning as commonly understood by one of ordinary skill in the art to which the instant specification pertains. Although any methods and materials similar or equivalent to those described herein can be used in the practice for testing of the instant specification, selected materials and methods are described herein. In describing and claiming the instant specification, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20% or ±10%, more preferably +5%, even more preferably +1%, and still more preferably +0.1% from the specified value, as such variations are appropriate to perform the disclosed methods.

A disease or disorder is “alleviated” if the severity of a symptom of the disease or disorder, the frequency with which such a symptom is experienced by a patient, or both, is reduced.

As used herein, the term “composition” or “pharmaceutical composition” refers to a mixture of at least one compound useful within the specification with a pharmaceutically acceptable carrier. The pharmaceutical composition facilitates administration of the compound to a patient or subject. Multiple techniques of administering a compound exist in the art including, but not limited to, intravenous, subcutaneous, oral, aerosol, parenteral, ophthalmic, pulmonary and topical administration.

An “effective amount” or “therapeutically effective amount” of a compound is that amount of compound that is sufficient to provide a beneficial effect to the subject to which the compound is administered. An “effective amount” of a delivery vehicle is that amount sufficient to effectively bind or deliver a compound.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the specification within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the specification, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the specification, and are physiologically acceptable to the patient. Supplementary active compounds may also be incorporated into the compositions. The “pharmaceutically acceptable carrier” may further include a pharmaceutically acceptable salt of the compound useful within the instant specification. Other additional ingredients that may be included in the pharmaceutical compositions used in the practice of the instant specification are known in the art and described, for example in Remington's Pharmaceutical Sciences (Genaro, Ed., Mack Publishing Co., 1985, Easton, PA), which is incorporated herein by reference.

As used herein, “treating a disease or disorder” means reducing the frequency with which a symptom of the disease or disorder is experienced by a patient. Disease and disorder are used interchangeably herein.

As used herein, the term “treatment” or “treating” encompasses therapy. Accordingly, the compositions and methods of the instant specification include therapeutic applications. Therefore “treating” or “treatment” of a state, disorder or condition includes: (i) inhibiting the state, disorder or condition, i.e., arresting or reducing the development of the disease or at least one clinical or subclinical symptom thereof, and/or (iii) relieving the disease, i.e. causing regression of the state, disorder or condition or at least one of its clinical or subclinical symptoms.

As used herein, the term “prevention” or “preventing” encompasses prophylaxis y. Accordingly, the compositions and methods of the instant specification include prophylactic applications. Therefore prevention” of or “preventing” a state, disorder or condition includes preventing or delaying the appearance of clinical symptoms of the state, disorder or condition developing in a subject that may be afflicted with or predisposed to the state, disorder or condition but does not yet experience or display clinical or subclinical symptoms of the state, disorder or condition.

Ranges: throughout this disclosure, various aspects can be presented in a range format. It should be understood that the description in range format is merely for convenience and brevity and should not be construed as an inflexible limitation on the scope of the instant specification. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Methods

The present disclosure provides a method of treating, ameliorating, and/or preventing post-operative pain in a subject.

In certain embodiments, the method comprises administering to the subject a therapeutically effective of a composition comprising at least one small extracellular vesicle (sEV) or exosome released by a macrophage.

In certain embodiments, the at least one sEV or exosome is derived from the macrophage using lipopolysaccharide (LPS) stimulation.

In certain embodiments, the at least one sEV or exosome is not derived from the macrophage using lipopolysaccharide (LPS) stimulation.

In certain embodiments, the composition is administered to the subject before the operation takes place.

In certain embodiments, the composition is administered to the subject at least one day before the operation takes place.

In certain embodiments, the subject is further administered at least one additional analgesic drug.

In certain embodiments, the amount of the at least one additional analgesic drug administered to the subject along with the composition is lower than the amount of the at least one analgesic drug that needs to be administered to the subject in the absence of the composition to provide a similar level of pain relief.

In certain embodiments, the duration of the pain relief provided by the at least one additional analgesic drug administered to the subject along with the composition is longer than the duration of the pain relief provided by the same amount of at least one additional analgesic drug administered in the absence of the composition.

In certain embodiments, the macrophage is allogenic with respect to the subject.

In certain embodiments, the macrophage is syngenic with respect to the subject.

### Method of Reducing Pain Caused by Physical Wound

In some aspects, the present invention is directed to a method of reducing a pain in response to a physical wound in a subject.

In some embodiments, the physical wound is caused by a surgical operation. In some embodiments, the pain is a post-operative pain.

In some embodiments, method comprising administering to the subject a therapeutically effective of a composition comprising a small extracellular vesicle (sEV) or an exosome released by a macrophage.

In some embodiments, the macrophage is situated in a medium, and releases the sEV or exosome into the medium. In some embodiments, the sEV or exosome is concentrated from the medium.

In some embodiments, the sEV or exosome is released by a macrophage subjected to a lipopolysaccharide (LPS) stimulation.

In some embodiments, the sEV or exosome is released by a macrophage not subjected to a lipopolysaccharide (LPS) stimulation.

In some embodiments, the composition further comprises a carrier. In some embodiments, the carrier is a pharmaceutically acceptable carrier.

In some embodiments, the composition is administered to the subject before, during or after the physical wound, such as the surgical operation, takes place.

In some embodiments, the composition is administered to the subject on the same day the physical wound, such as the surgical operation, takes place.

In some embodiments, the composition is administered to the subject 1 day before, 2 days before, 3 days before, 4 days before, 5 days before, 6 days before, 7 days before, 2 weeks before, or more than 2 weeks before the physical wound, such as the surgical operation, takes place, or any time range therebetween.

In some embodiments, the composition is administered 1 day after, 2 days after, 3 days after, 4 days after, 5 days after, 6 days after, 7 days after, 2 weeks after, or more than 2 weeks after the physical wound, such as the surgical operation, takes place, or any time range therebetween.

In some embodiments, the composition is administered multiple times across a time period.

In some embodiments, the administration of the composition starts before the physical wound takes place up till when the physical wound takes place or after the physical wound takes place.

In some embodiments, the administration of the composition starts from the physical wound takes place and is continued for a time until certain time after the physical wound takes place.

In some embodiments, the time at which the physical wound is received cannot be predicted, such as the case of a solider in a combat situation, or a person working in a hazardous environment and thereby subjected to the possibility of physical injury. In some embodiments, the administration of the composition starts when the possibility of the of the subject to receive a physical wound becomes a non-trivial. It is worth noting that, unlike pain managing compounds like opioids or ketamine, the composition herein is not expected to cause addiction.

In some embodiments, the subject is further administered at least one additional analgesic drug. Non-limiting examples of the additional analgesic drug include acetaminophen; Nonsteroidal anti-inflammatory drugs (NSAID) such as aspirin, ibuprofen, naproxen, and the like; ketamine; and the like.

In some embodiments, the amount of the at least one additional analgesic drug administered to the subject along with the composition is lower than the amount of the at least one analgesic drug that needs to be administered to the subject in the absence of the composition to provide a similar level of post-operative pain relief.

In some embodiments, the duration of the pain relief provided by the amount of the at least one additional analgesic drug administered to the subject along with the composition is longer than the duration of the pain relief provided by the same amount of at least one additional analgesic drug administered in the absence of the composition.

In some embodiments, the macrophage is allogenic with respect to the subject.

In some embodiments, the macrophage is syngenic with respect to the subject.

In some embodiments, the macrophage is a macrophage cell-line.

In some embodiments, the macrophage is obtained from the subject. In some embodiments, before, during, or after receiving the physical wound, the macrophage (or progenitor cells differentiate into the same) is obtained from the subject, and sEVs or exosomes are made using the macrophage, such as with or without the LPS stimulation.

In some embodiments, the macrophage is obtained from a donated blood. When blood banks receive blood from a donor, blood factions containing white blood cells such as macrophages and progenitor cells thereof are often discarded. In some embodiments, these otherwise discarded macrophages and progenitor cells are used to prepare the sEVs and the exosomes.

In some embodiments, the sEVs and the exosomes are collected from a medium the macrophage situate in. In some embodiments, the sEVs and the exosomes are collected from the medium by an ultracentrifugation method.

In some embodiments, the sEV or exosome is administered to the subject parentally.

In some embodiments, the subject is a mammal. In some embodiments, the subject is a human.

### Method of Performing Surgical Operation

In some aspects, the present invention is directed to a method of performing a surgical operation on a subject.

In some embodiments, the method comprises performing a surgery on the subject; and administering to the subject an effective amount of a composition comprising a small extracellular vesicle (sEV) or an exosome released by a macrophage.

In some embodiments, the sEV or exosome reduces a post-operative pain caused by the surgery.

In some embodiments, the sEV, exosome, composition, and administration of the composition are the same as or similar to those as described elsewhere herein, such as in the “Method of Reducing Pain Caused by Physical Wound” section.

### Combination Therapies

In some embodiments, the method of reducing pain caused by physical wound, the method of treating, ameliorating, and/or preventing the post-operative pain, or the performing surgical operations comprise administering to the subject the effective amount of at least one compound and/or composition contemplated within the disclosure.

In some embodiments, the composition herein includes at least one compound and/or composition contemplated within the disclosure.

In some embodiments, the subject is further administered at least one additional agent that treats, ameliorates, and/or prevents a disease and/or disorder contemplated herein. In other embodiments, the compound and the at least one additional agent are co-administered to the subject. In yet other embodiments, the compound and the at least one additional agent are co-formulated.

The compounds contemplated within the disclosure are intended to be useful in combination with one or more additional compounds. These additional compounds may comprise compounds of the present disclosure and/or at least one additional agent for treating or reducing pain, and/or at least one additional agent that treats one or more diseases or disorders contemplated herein.

A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations contemplated within the disclosure may be administered to the subject either prior to or after the onset of a disease and/or disorder contemplated herein. Further, several divided dosages, as well as staggered dosages may be administered daily or sequentially, or the dose may be continuously infused, or may be a bolus injection. Further, the dosages of the therapeutic formulations contemplated within the disclosure may be proportionally increased or decreased as indicated by the exigencies of the therapeutic or prophylactic situation.

Administration of the compositions contemplated within the disclosure to a patient, preferably a mammal, more preferably a human, may be carried out using known procedures, at dosages and for periods of time effective to treat a disease and/or disorder contemplated herein in the patient. An effective amount of the therapeutic compound necessary to achieve a therapeutic effect may vary according to factors such as the state of the disease or disorder in the patient; the age, sex, and weight of the patient; and the ability of the therapeutic compound contemplated within the disclosure to treat a disease and/or disorder contemplated herein in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound contemplated within the disclosure is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions contemplated within the disclosure may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds contemplated within the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms contemplated within the disclosure are dictated by and directly dependent on (a) the unique characteristics of the therapeutic compound and the particular therapeutic effect to be achieved, and (b) the limitations inherent in the art of compounding/formulating such a therapeutic compound for the treatment of a disease and/or disorder contemplated herein.

In certain embodiments, the compositions of the disclosure are formulated using one or more pharmaceutically acceptable excipients or carriers. In certain embodiments, the pharmaceutical compositions of the disclosure comprise a therapeutically effective amount of a compound of the disclosure and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions of the disclosure are administered to the patient in dosages that range from one to five times per day or more. In another embodiment, the compositions of the disclosure are administered to the patient in range of dosages that include, but are not limited to, once every day, every two, days, every three days to once a week, and once every two weeks. It is readily apparent to one skilled in the art that the frequency of administration of the various combination compositions of the disclosure varies from individual to individual depending on many factors including, but not limited to, age, disease or disorder to be treated, gender, overall health, and other factors. Thus, the disclosure should not be construed to be limited to any particular dosage regime and the precise dosage and composition to be administered to any patient is determined by the attending physical taking all other factors about the patient into account.

Compounds of the disclosure for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 g to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the disclosure is from about 1 mg and about 2,500 mg. In some embodiments, a dose of a compound of the disclosure used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce pain in a patient.

Formulations may be employed in admixtures with conventional excipients, i.e., pharmaceutically acceptable organic or inorganic carrier substances suitable for intracranially, intrathecal, oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the disclosure include oral, nasal, rectal, intravaginal, parenteral, buccal, sublingual or topical. The compounds for use in the disclosure may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the disclosure may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

The present disclosure also includes a multi-layer tablet comprising a layer providing for the delayed release of one or more compounds of the disclosure, and a further layer providing for the immediate release of another medication. Using a wax/pH-sensitive polymer mix, a gastric insoluble composition may be obtained in which the active ingredient is entrapped, ensuring its delayed release.

### Parenteral Administration

For parenteral administration, the compounds of the disclosure may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this disclosure include dosage forms as described in U.S. Pat. Nos. 6,340,475; 6,488,962; 6,451,808; 5,972,389; 5,582,837; and 5,007,790. Additional dosage forms of this disclosure also include dosage forms as described in U.S. Patent Applications Nos. 20030147952; 20030104062; 20030104053; 20030044466; 20030039688; and 20020051820. Additional dosage forms of this disclosure also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present disclosure may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the disclosure may be administered in the form of microparticles, for example, by injection or in the form of wafers or discs by implantation.

In certain embodiments of the disclosure, the compounds of the disclosure are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present disclosure depends on the age, sex and weight of the patient, and the current medical condition of the patient. The skilled artisan is able to determine appropriate dosages depending on these and other factors.

A suitable dose of a compound of the present disclosure may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the modulator of the disclosure is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the patient's condition, to a level at which the improved disease is retained. In certain embodiments, patients require intermittent treatment on a long-term basis upon any recurrence of symptoms and/or infection.

The compounds for use in the method of the disclosure may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. Capsid assembly modulators exhibiting high therapeutic indices are preferred. The data obtained from cell culture assays and animal studies are optionally used in formulating a range of dosage for use in human. The dosage of such capsid assembly modulators lies preferably within a range of circulating concentrations that include the ED₅₀ with minimal toxicity. The dosage optionally varies within this range depending upon the dosage form employed and the route of administration utilized.

Those skilled in the art recognizes, or is able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in assay and/or reaction conditions, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1-1

Surgeries induce post-operative pain that must be alleviated effectively to reduce suffering, and to promote healing and recovery. Depending on the surgery and the specific conditions, post-operative pain is not effectively managed in >80% of the patients in US. Traditional opioids, such as morphine, remain the standard of care for perioperative pain regulation. Limitations of opioid use include a narrow therapeutic window, undesirable adverse events, and toxicity. Better treatment strategies are needed during and immediately after surgery to prevent the progression of acute pain to long term persistent pain. Small extracellular vesicles (sEVs), including exosomes, mediate intercellular communication by carrying biomolecular cargo to recipient cells. sEVs isolated from macrophage derived RAW 264.7 cells attenuate pain and inflammation in a mouse model of inflammatory pain. In certain embodiments, macrophage-derived sEVs can attenuate post-operative pain therapeutically (administered immediately after surgery) or prophylactically (administered 2 weeks prior to surgery), decreasing the dose and duration of analgesic needed for pain relief.

In the study described herein (the present study), sEVs released by RAW 264.7 cells treated with or without LPS (Exo+ or Exo− respectively) were isolated by ultracentrifugation. Male C57BL/6J mice underwent paw incisional surgeries and either 5 or 10 μg Exo+, Exo− or PBS was administered either via intrathecal or intraplantar injection for 5 days following injury/injection, behavior was assessed via von Frey and Hargreaves to test mechanical and thermal sensitivity, respectively. At various time points, mice were sacrificed, and their paw tissue was collected for IHC and flow cytometry to assess immune cell trafficking.

The present study generated and validated the model and the data show that 5 μg intrathecal injections of Exo+ and Exo− sEVs after surgery results in faster attenuation of mechanical and thermal hypersensitivity compared to control or mice that received sEVs by intraplantar injection.

The data show that intrathecal injections of 10 μg Exo+/− are therapeutic for postoperative pain.

### Example 1-2

Whether sEVs have a prophylactic effect is tested. The synergistic effect of therapeutic and prophylactic sEVs is assessed to show that sEVs can a) lower the efficacious dose of commonly prescribed analgesic drugs and b) increase the duration of pain relief in a post-operative pain model.

### Example 1-3: Materials and Methods

### RAW 264.7 Mouse Macrophage Cell Line Culture

RAW 264.7 mouse macrophage cells (ATCC TIB-71) were cultured in complete DMEM (Sigma-Aldrich) supplemented with 10% heat-inactivated FBS (Gibco), 100 U/mL penicillin-streptomycin (Gibco) at 37° C. with 5% CO₂. At 70-80% confluence, cells were washed with PBS without ions (Gibco) and the medium was changed to DMEM medium supplemented with 10% exosome-deplete heat inactivated FBS, 100 U/mL penicillin-streptomycin in the presence or absence of 1 μg/ml LPS (Sigma-Aldrich) at 37° C. with 5% CO₂. Conditioned media was collected at 24 hours for sEVs isolation.

Isolation and Purification of sEVs from RAW 264.7 Cell Conditioned Media

sEVs were purified as described in McDonald et al. (Journal of visualized experiments: JoVE, e50294) and McDonald et al. (PAIN 155, 1527-1539) with some modification. Cells were grown in exosome-deplete media for 24 hours. Conditioned media was collected and centrifuged at 300×g for 10 min at 4° C. to pellet dead cells followed by centrifugation at 12,000×g for 30 min at 4° C. to remove debris and large vesicles. The supernatant was filtered through a 0.22 μm syringe filter and 12 ml of filtered supernatant was concentrated to a final volume of 500 μL with 100 kDa concentrator (Amicon Millipore Sigma, Cat: ACS510024) by centrifugation at 5000×g at 4° C. for 30 minutes in a fixed angle rotor (Beckman Coulter, Brea, CA). The qEV size exclusion chromatography column (Izon product Code: SP5) was washed using 1× PBS and 500 μl of concentrated supernatant was then loaded onto the qEV size exclusion chromatography column followed by 2.5 mL PBS to collect 3 ml void volume. When the column flow stopped, 2 ml of PBS was added to collect EV zone sample. The EV volume (2 ml) was then centrifuged for 70 minutes at 45,000×g at 4° C. The EV pellet was then resuspended in DPBS and stored in −20° C. Protein was determined using micro BCA.

### Mice

Behavioral tests were performed using C57BL/6J mice (8-10 weeks old) obtained from Jackson Laboratories. Mice were housed in 12 h light/dark cycles. Behavioral assays were performed by researchers blinded to the treatment received.

### Example 2-1: Macrophage Derived Small Extracellular Vesicles as a Therapeutic for Postoperative Pain and Inflammation

Surgeries induce post-operative pain that must be alleviated effectively to reduce suffering, and to promote healing and recovery. Depending on the surgery and the specific conditions, post-operative pain is not effectively managed in >80% of the patients in US. Traditional opioids, such as morphine, remain the standard of care for perioperative pain regulation. Limitations of opioid use include a narrow therapeutic window, undesirable adverse events, and toxicity. Better treatment strategies are needed during and immediately after surgery to prevent the progression of acute pain to long term persistent pain. Small extracellular vesicles (sEVs), including exosomes, mediate intercellular communication by carrying biomolecular cargo to recipient cells.

It has been shown that sEVs isolated from macrophage derived RAW 264.7 cells attenuate pain and inflammation in a mouse model of inflammatory pain. However, due to significant differences between post-operative pain and inflammatory pain, it was not known in the art whether such sEVs are effective in the attenuation of post-operative pain.

The present study tested whether macrophage-derived sEVs can attenuate post-operative pain therapeutically (administered immediately after surgery) or prophylactically (administered 2 weeks prior to surgery), thus decreasing the dose and duration of analgesic needed for pain relie. The present study generated and validated the model and the preliminary data revealed that 5 μg intrathecal injections of sEVs after surgery results in faster attenuation of mechanical hypersensitivity compared to control.

### Example 2-2: Methods

### Cell Culture

Media from macrophage derived RAW 264.7 cells cultured in Exo deplete media±LPS were used (Exo+ with LPS; Exo− without LPS stimulation).

### sEV Isolation

Exo(−) or Exo(+) sEVs were isolated by ultracentrifugation and qEV size exclusion chromatography.

### Behavioral Methods

von Frey was used to test mechanical pain threshold; Hargreaves test was used for thermal pain thresholds.

### Surgery

A longitudinal incision was made through the muscle belly of the elevated flexor digitorum brevis muscle from proximal to distal ends of the cutaneous incision and curved forceps were inserted under the flexor digitorum brevis muscle to elevate the muscle.

### Example 2-3: Validation of the Post Operative Pain Model

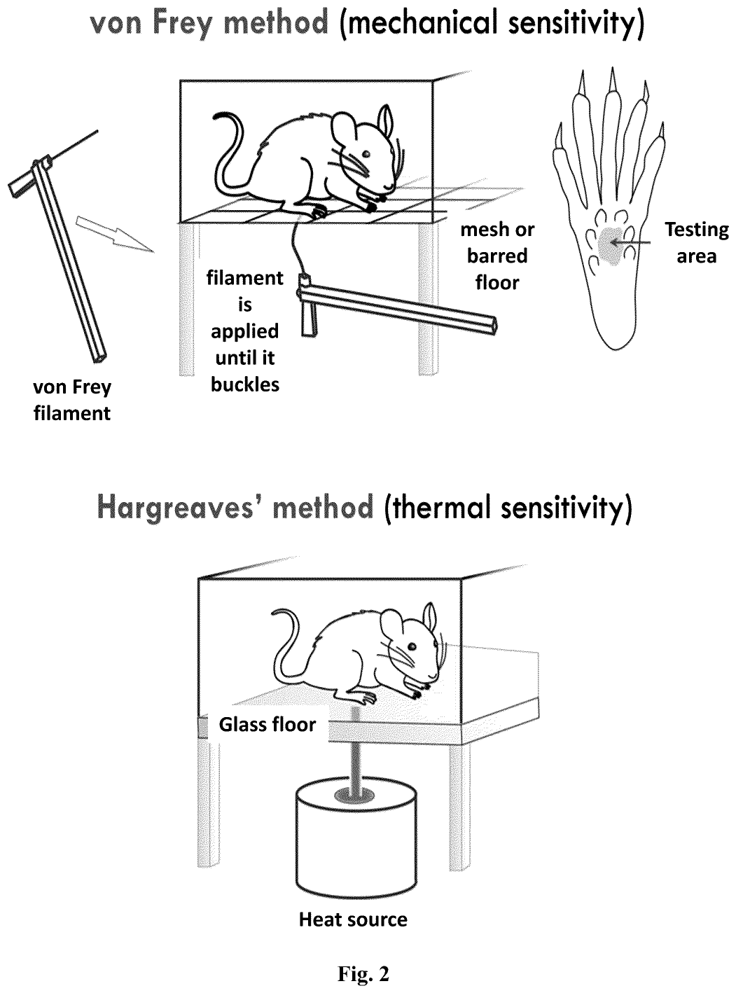

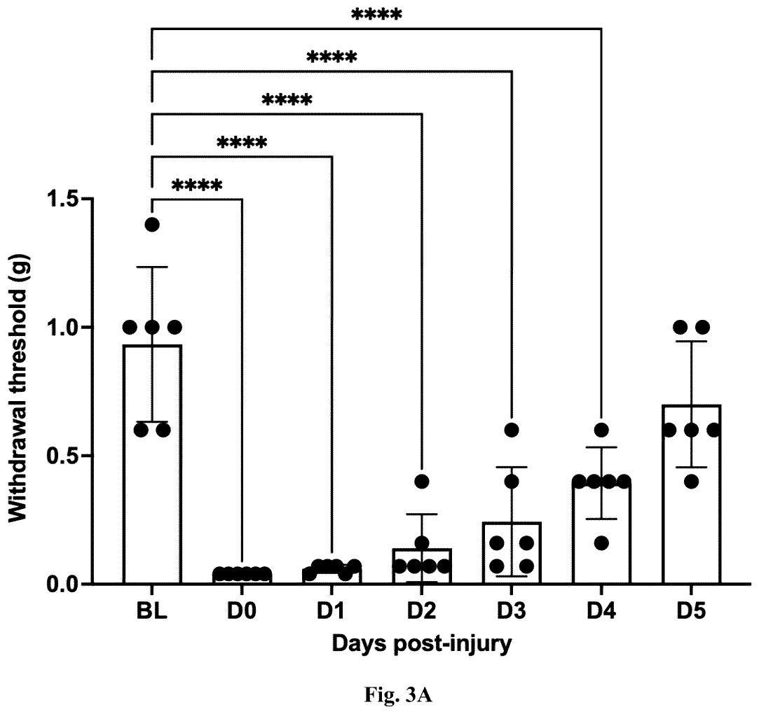

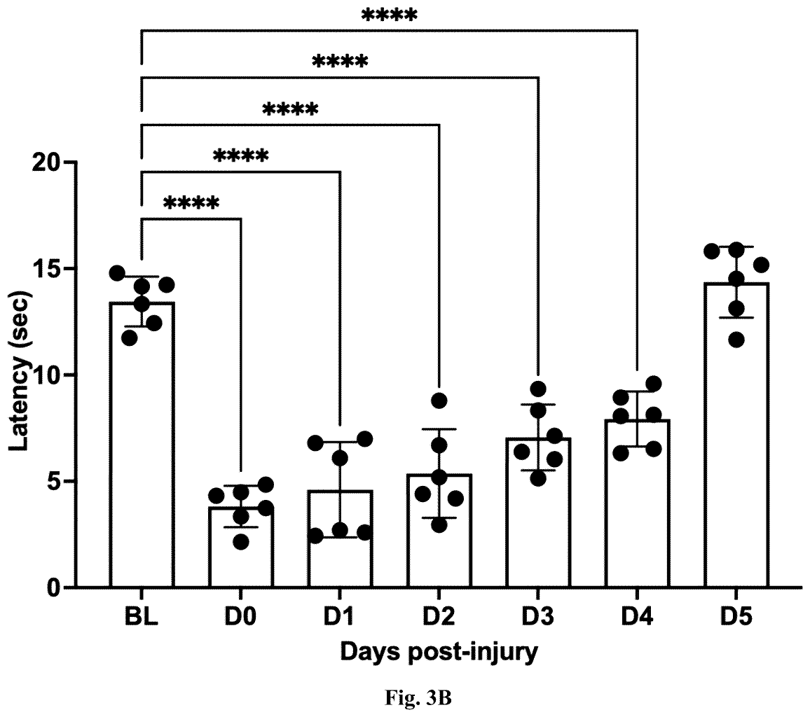

Referring to FIGS. 3A-3B, both the von Frey test of mechanical sensitivity and the Hargreaves's test of thermal sensitivity (see FIG. 2) work with the post operative pain model herein. Specifically, the incision caused the significantly reduced withdrawal threshold in the von Frey test and significantly reduced latency in withdrawal in the Hargreaves's test. The allodynia (i.e., sensation of pain due to a stimulus that does not normally provoke pain) observed in the tests confirmed the establishment of post-incision model. Both the withdrawal threshold and the latency gradually increased in the days post injury, as the pain went away.

### Example 2-4: Therapeutic Administrations of the sEVs/Exosome were Effective in Attenuating Post Operative Pain

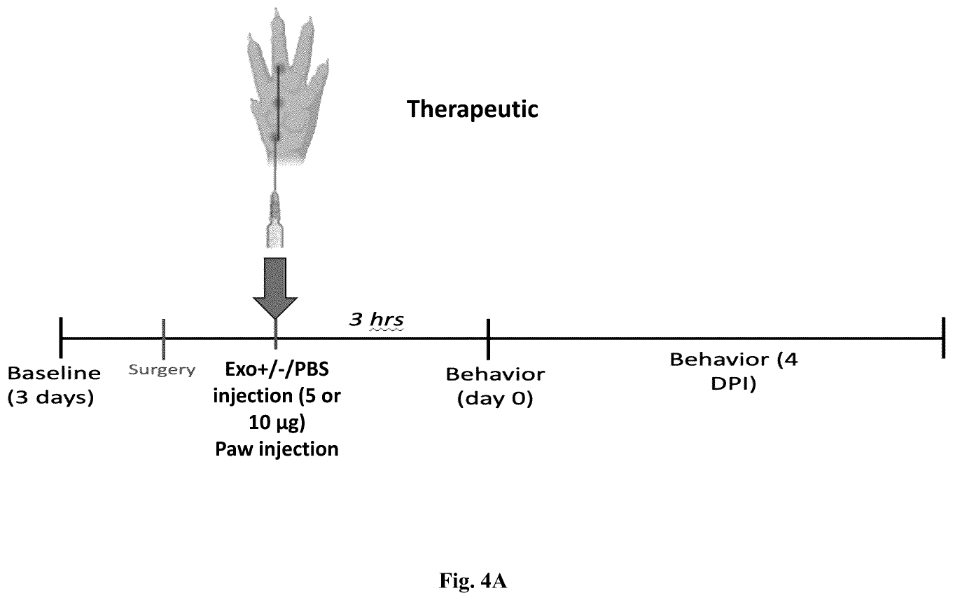

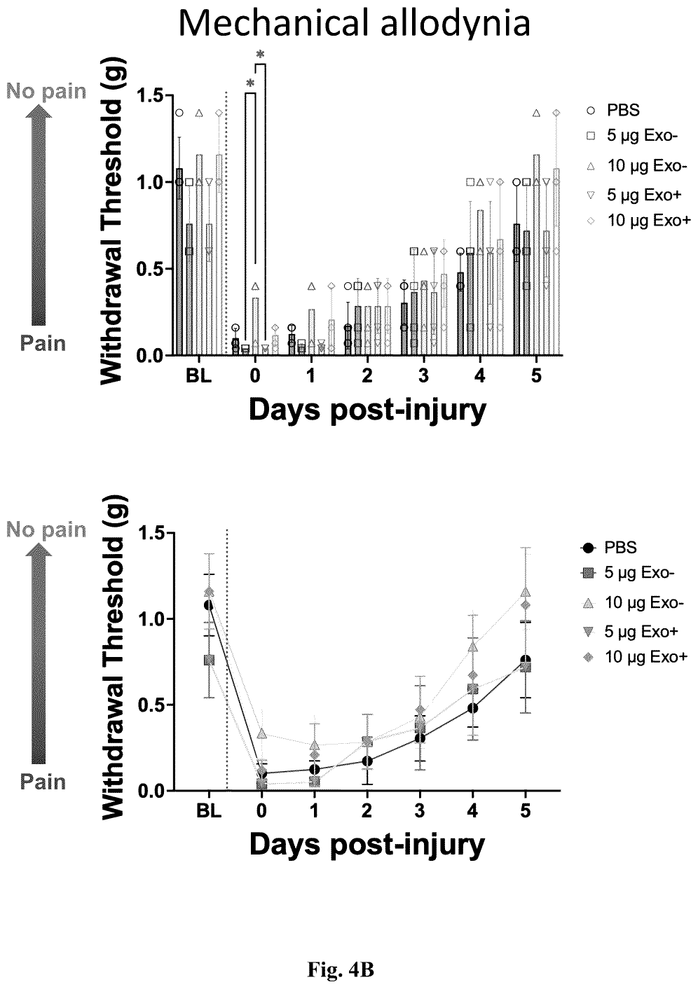

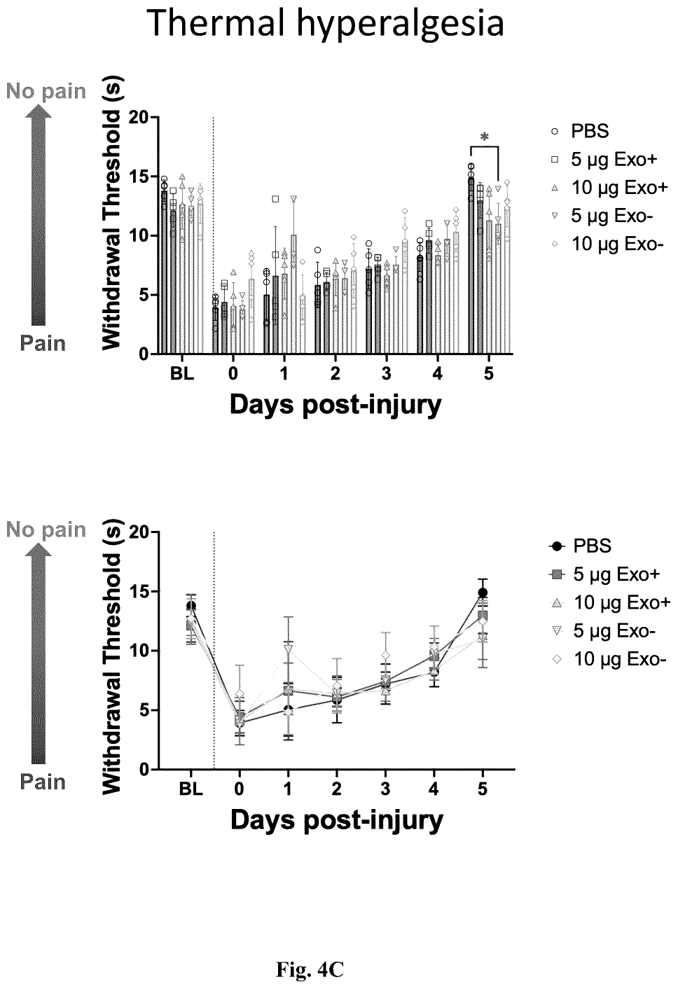

Referring to FIGS. 4A-4C, the therapeutic intraplantar injections of sEVs (Exo+/−) attenuate pain hypersensitivity in a dose dependent manner in the paw incisional model.

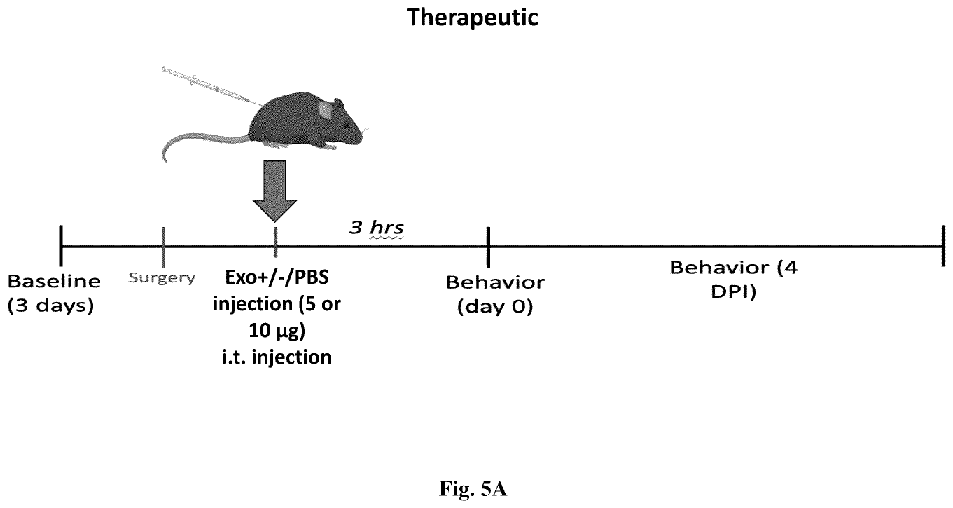

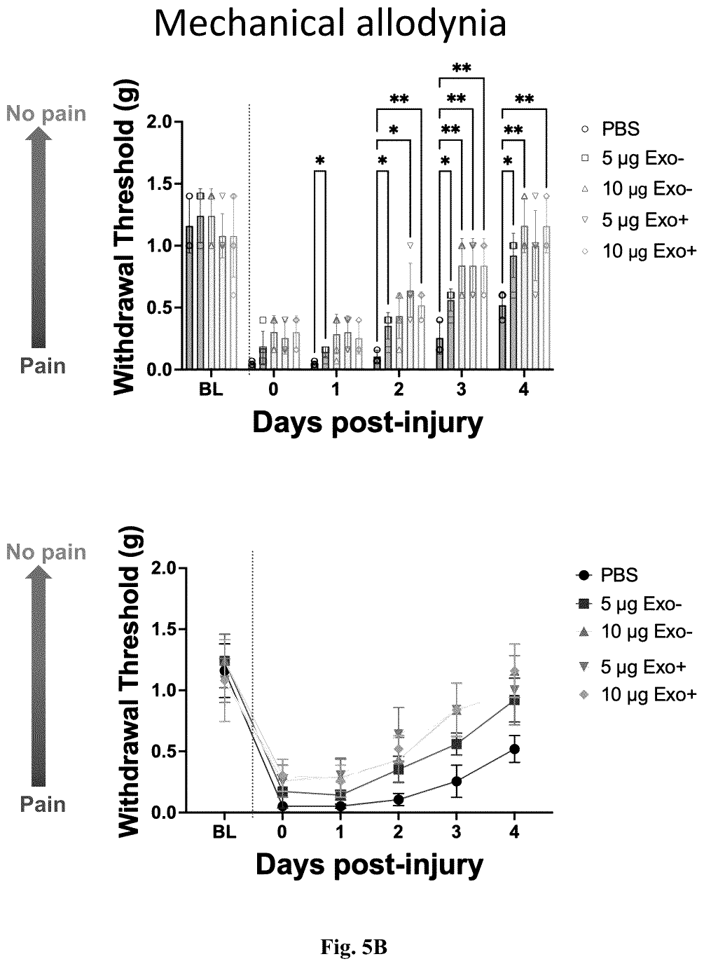

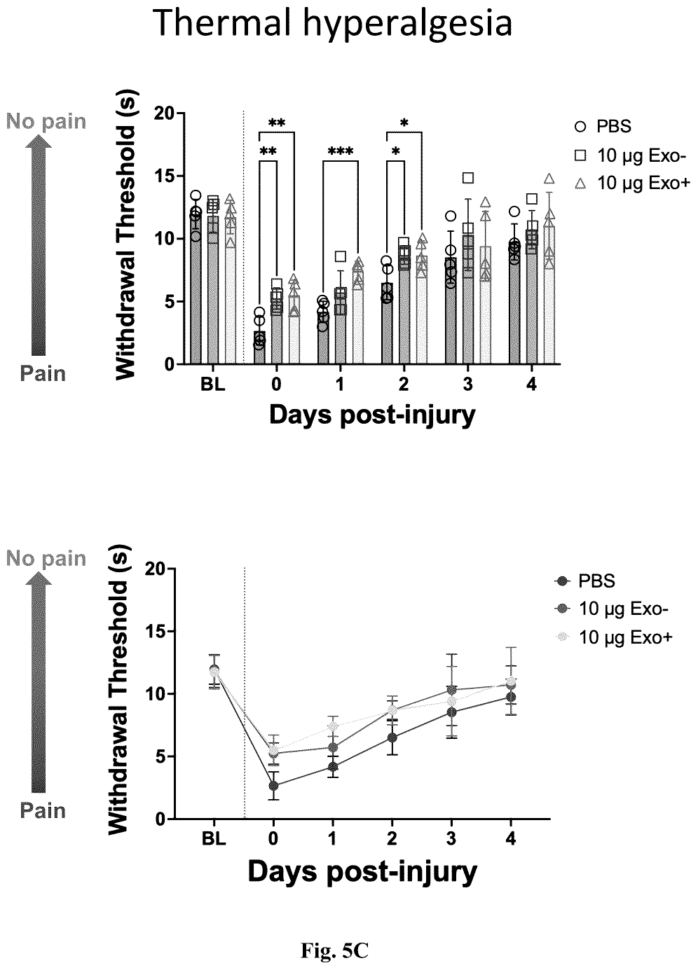

Referring to FIGS. 5A-5C, the therapeutic intrathecal injections of sEVs (Exo+/−) attenuate pain in a dose dependent manner in the paw incisional model.

### Example 2-5: Prophylactic Administrations of the sEVs/Exosome were Effective in Attenuating Post Operative Pain

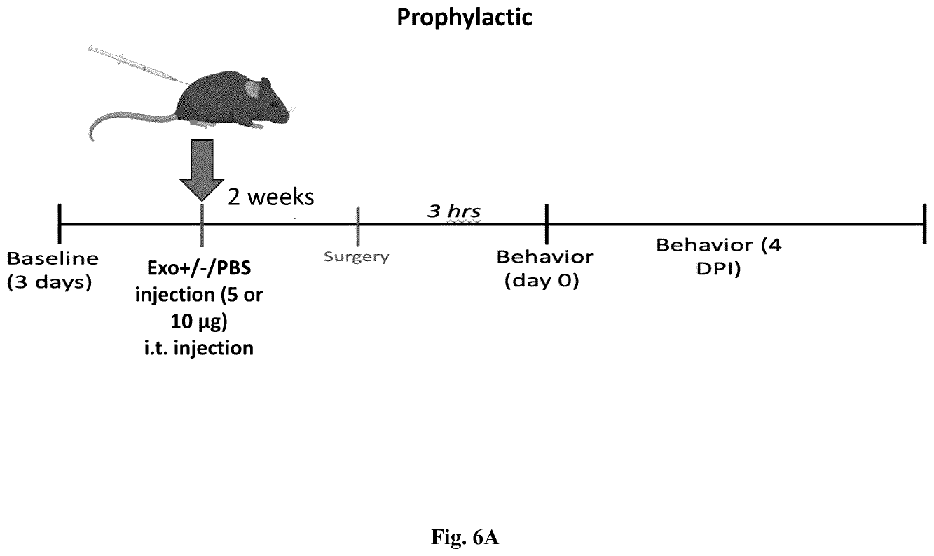

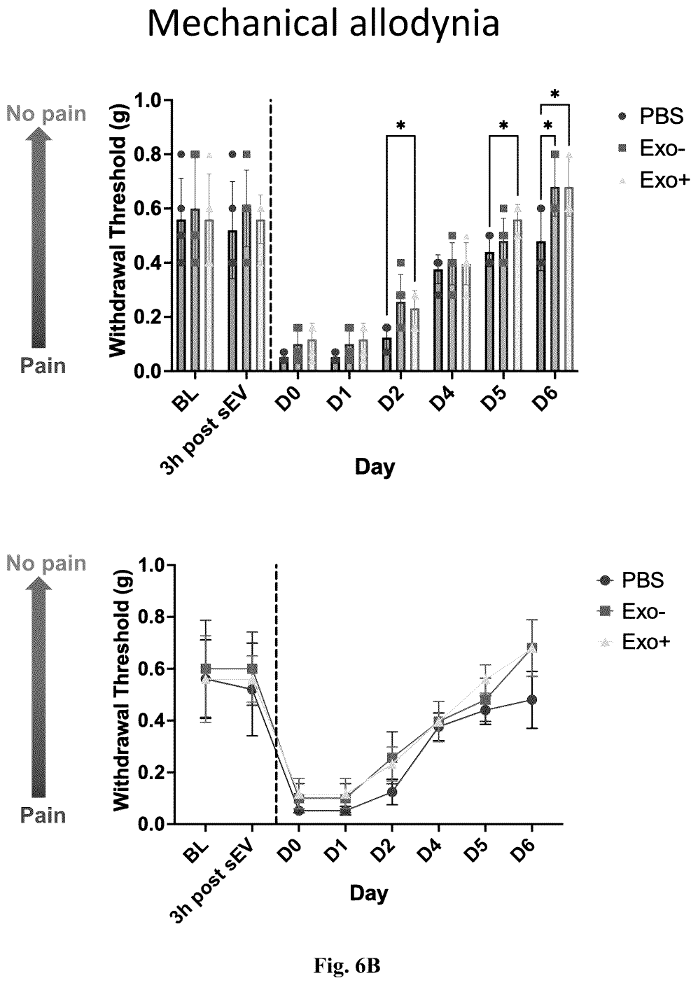

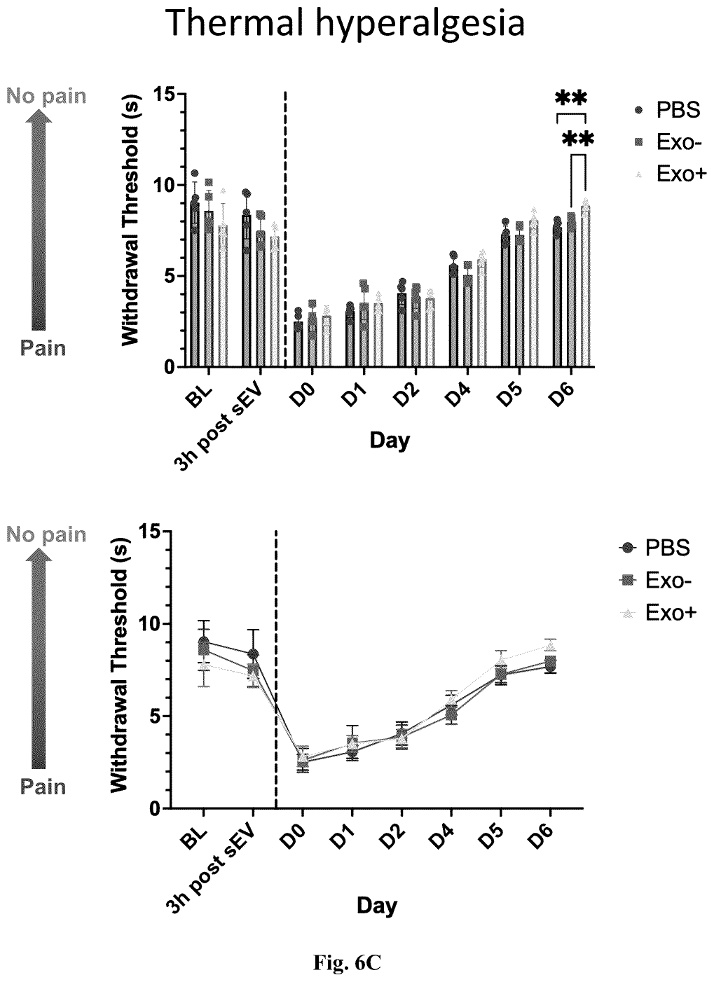

Referring to FIGS. 6A-6C, the rophylactic intrathecal injections of sEVs (Exo+/−) attenuate pain hypersensitivity in the paw incision model of postoperative pain.

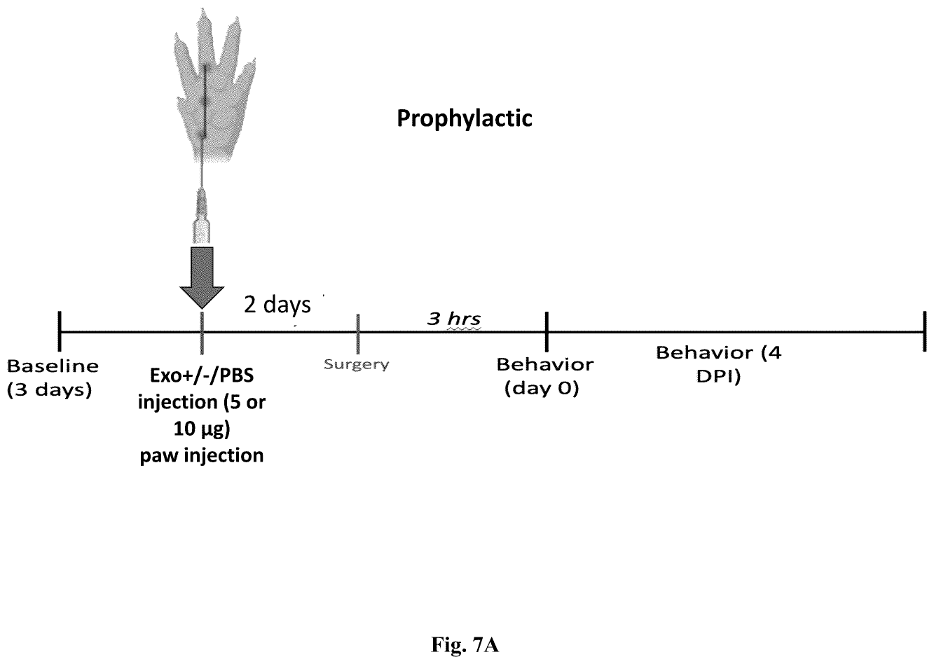

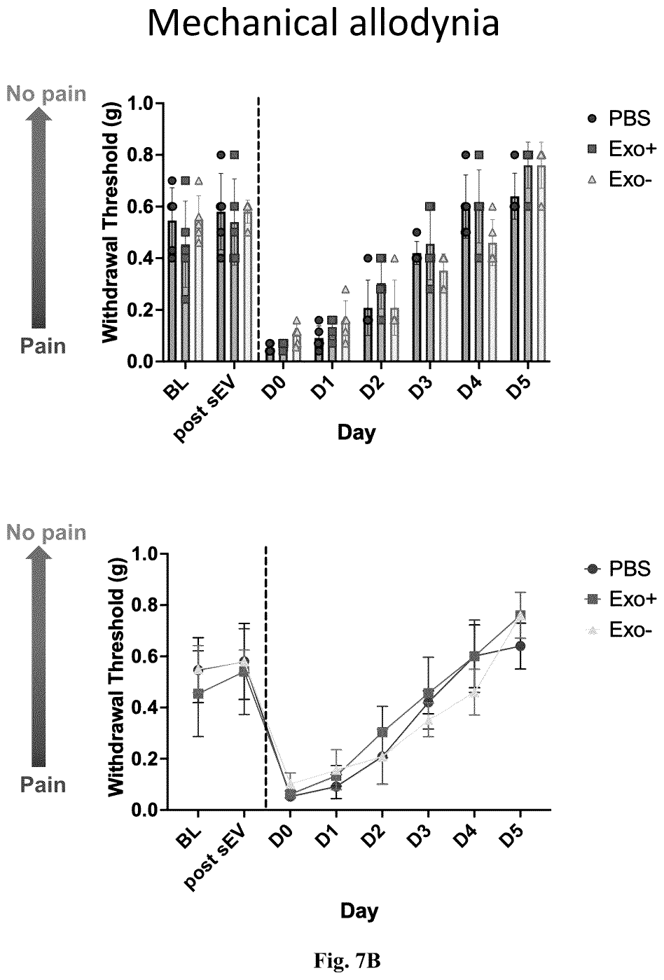

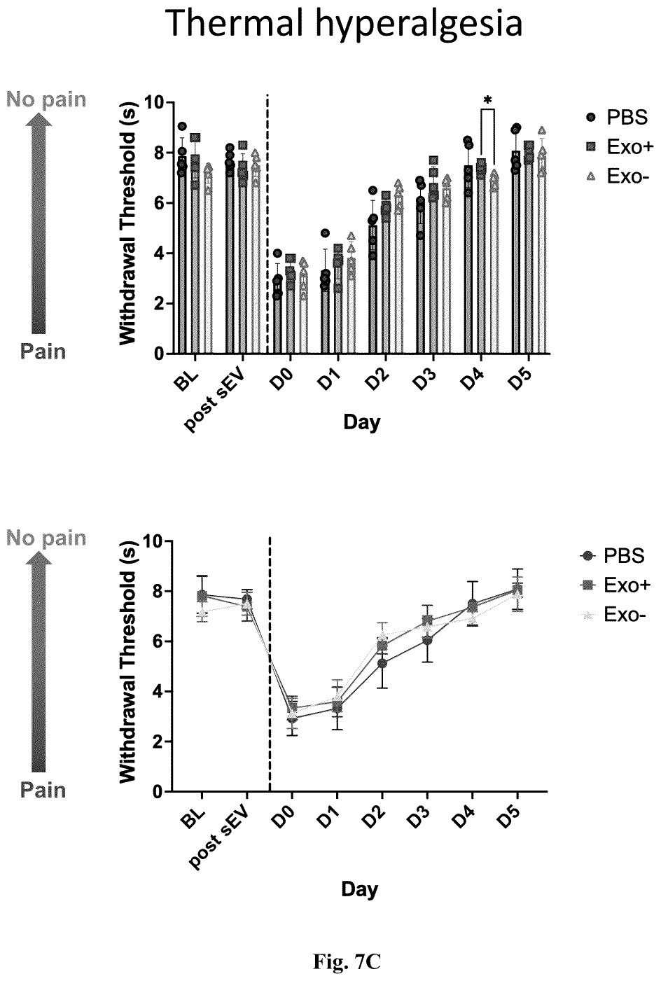

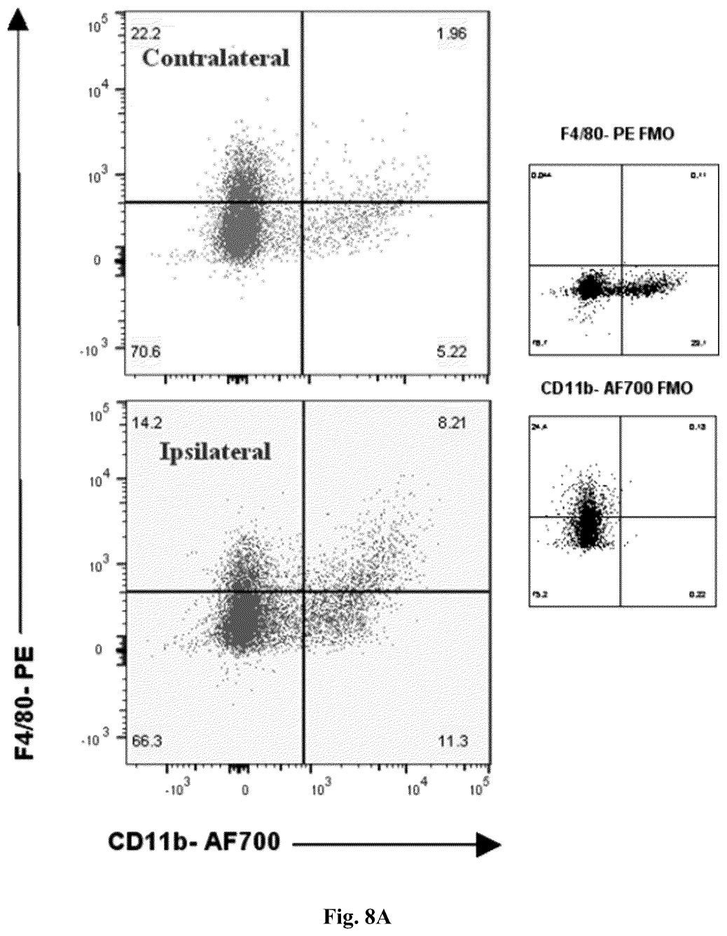

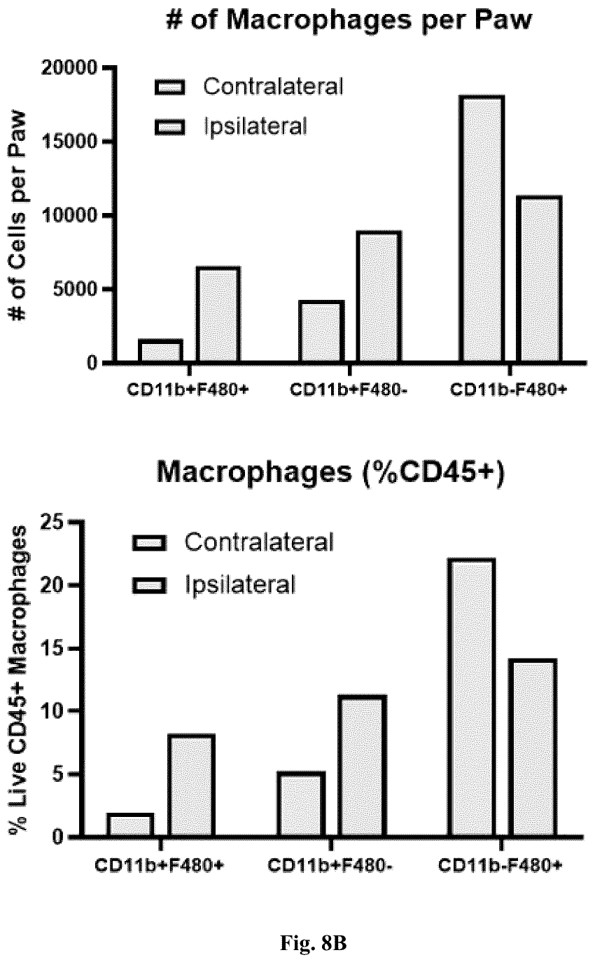

Referring to FIGS. 7A-7C, prophylactic intraplantar injections of sEVs (Exo+/−) also has effect on pain hypersensitivity in the paw incision model of postoperative pain.

### Example 2-6

In the present study, a post-incision model was developed for the study of post-operative pain. The validity of this model was confirmed by allodynia tests.

sEVs showed a dose dependent effect in attenuating pain hypersensitivity.

The administrations of sEVs both after the incision (“therapeutic”) and before the incision (“prophylactic”) were confirmed to be effective in attenuating the pain caused by the incision.

The present study discovered that the intrathecal injections of sEVs were more efficacious in attenuating mechanical allodynia and thermal hyperalgesia when compared to an intraplantar sEV injection. Without wishing to be bound by theory, it is hypothesized that the leakage of sEVs from the paw incision site contributed to the reduced efficacy.

### Enumerated Embodiments

In some aspects, the present invention is directed to the following non-limiting embodiments:

Embodiment 1: A method of treating, ameliorating, and/or preventing post-operative pain in a subject, the method comprising administering to the subject a therapeutically effective of a composition comprising a small extracellular vesicle (sEV) or an exosome released by a macrophage.

Embodiment 2: The method of Embodiment 1, wherein at least one of the following applies:
(a) the sEV or exosome is derived from the macrophage using lipopolysaccharide (LPS) stimulation,
(b) the sEV or exosome is derived from the macrophage without using lipopolysaccharide (LPS) stimulation.

Embodiment 3: The method of Embodiment 1, wherein the composition is administered to the subject before, during or after the operation takes place.

Embodiment 4: The method of Embodiment 1, wherein at least one of the following applies:
(a) the composition is administered to the subject at least 1 day, 2 days, 3 days, 4 days, 5 days, 6 days, 7 days, 2 weeks, or more than 2 weeks before the operation takes place,
(b) the composition is administered at least 1 day, 2 days, 3 days, 4 days, 5 days, 6 days, 7 days, 2 weeks, or more than 2 weeks after the operation takes place.

Embodiment 5: The method of Embodiment 1, wherein the subject is further administered at least one additional analgesic drug.

Embodiment 6: The method of Embodiment 5, wherein at least one of the following applies:
(a) the amount of the at least one additional analgesic drug administered to the subject along with the composition is lower than the amount of the at least one analgesic drug that needs to be administered to the subject in the absence of the composition to provide a similar level of post-operative pain relief,
(b) the duration of the pain relief provided by the amount of the at least one additional analgesic drug administered to the subject along with the composition is longer than the duration of the pain relief provided by the same amount of at least one additional analgesic drug administered in the absence of the composition.

Embodiment 7: The method of Embodiment 1, wherein the macrophage is allogenic with respect to the subject, or the macrophage is syngenic with respect to the subject.

Embodiment 8: The method of Embodiment 1, wherein the macrophage is obtained from the subject, or obtained from a donated blood.

Embodiment 9: The method of Embodiment 1, wherein the sEV or exosome is administered to the subject parentally.

Embodiment 10: The method of Embodiment 1, wherein the subject is a human.

Embodiment 11: A method of performing a surgical operation on a subject, the method comprising:
performing a surgery on the subject; and
administering to the subject an effective amount of a composition comprising a small extracellular vesicle (sEV) or an exosome released by a macrophage,
wherein the sEV or exosome reduces a post-operative pain caused by the surgery.

Embodiment 12: The method of Embodiment 11, wherein at least one of the following applies:
the sEV or exosome is derived from the macrophage using lipopolysaccharide (LPS) stimulation,
the sEV or exosome is derived from the macrophage without using lipopolysaccharide (LPS) stimulation.

Embodiment 13: The method of Embodiment 11, wherein the composition is administered to the subject before, during or after the surgery takes place.

Embodiment 14: The method of Embodiment 11, wherein at least one of the following applies:
(a) the composition is administered to the subject at least 1 day, 2 days, 3 days, 4 days, 5 days, 6 days, 7 days, 2 weeks, or more than 2 weeks before the surgery takes place,
(b) the composition is administered at least 1 day, 2 days, 3 days, 4 days, 5 days, 6 days, 7 days, 2 weeks, or more than 2 weeks after the surgery takes place.

Embodiment 15: The method of Embodiment 11, further comprising administering to the subject at least one additional analgesic drug.

Embodiment 16: The method of Embodiment 15, wherein at least one of the following applies:
(a) the amount of the at least one additional analgesic drug administered to the subject along with the composition is lower than the amount of the at least one analgesic drug that needs to be administered to the subject in the absence of the composition to provide a similar level of post-operative pain relief,
(b) the duration of the pain relief provided by the amount of the at least one additional analgesic drug administered to the subject along with the composition is longer than the duration of the pain relief provided by the same amount of at least one additional analgesic drug administered in the absence of the composition.

Embodiment 17: The method of Embodiment 11, wherein the macrophage is allogenic with respect to the subject, or the macrophage is syngenic with respect to the subject.

Embodiment 18: The method of Embodiment 11, wherein the macrophage is obtained from the subject, or obtained from a donated blood.

Embodiment 19: The method of Embodiment 11, wherein the sEV or exosome is administered to the subject parentally.

Embodiment 20: The method of claim 11, wherein the subject is a human.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A method of treating, ameliorating, and/or preventing post-operative pain in a subject, the method comprising administering to the subject a therapeutically effective of a composition comprising a small extracellular vesicle (sEV) or an exosome released by a macrophage.

2. The method of claim 1, wherein at least one of the following applies:
(a) the sEV or exosome is derived from the macrophage using lipopolysaccharide (LPS) stimulation,
(b) the sEV or exosome is derived from the macrophage without using lipopolysaccharide (LPS) stimulation.

3. The method of claim 1, wherein the composition is administered to the subject before, during or after the operation takes place.

4. The method of claim 1, wherein at least one of the following applies:
(a) the composition is administered to the subject at least 1 day, 2 days, 3 days, 4 days, 5 days, 6 days, 7 days, 2 weeks, or more than 2 weeks before the operation takes place,
(b) the composition is administered at least 1 day, 2 days, 3 days, 4 days, 5 days, 6 days, 7 days, 2 weeks, or more than 2 weeks after the operation takes place.

5. The method of claim 1, wherein the subject is further administered at least one additional analgesic drug.

6. The method of claim 5, wherein at least one of the following applies:
(a) the amount of the at least one additional analgesic drug administered to the subject along with the composition is lower than the amount of the at least one analgesic drug that needs to be administered to the subject in the absence of the composition to provide a similar level of post-operative pain relief,
(b) the duration of the pain relief provided by the amount of the at least one additional analgesic drug administered to the subject along with the composition is longer than the duration of the pain relief provided by the same amount of at least one additional analgesic drug administered in the absence of the composition.

7. The method of claim 1, wherein the macrophage is allogenic with respect to the subject, or the macrophage is syngenic with respect to the subject.

8. The method of claim 1, wherein the macrophage is obtained from the subject, or obtained from a donated blood.

9. The method of claim 1, wherein the sEV or exosome is administered to the subject parentally.

10. The method of claim 1, wherein the subject is a human.

11. A method of performing a surgical operation on a subject, the method comprising:
performing a surgery on the subject; and
administering to the subject an effective amount of a composition comprising a small extracellular vesicle (sEV) or an exosome released by a macrophage,
wherein the sEV or exosome reduces a post-operative pain caused by the surgery.

12. The method of claim 11, wherein at least one of the following applies:
(c) the sEV or exosome is derived from the macrophage using lipopolysaccharide (LPS) stimulation,
(d) the sEV or exosome is derived from the macrophage without using lipopolysaccharide (LPS) stimulation.

13. The method of claim 11, wherein the composition is administered to the subject before, during or after the surgery takes place.

14. The method of claim 11, wherein at least one of the following applies:
(a) the composition is administered to the subject at least 1 day, 2 days, 3 days, 4 days, 5 days, 6 days, 7 days, 2 weeks, or more than 2 weeks before the surgery takes place,
(b) the composition is administered at least 1 day, 2 days, 3 days, 4 days, 5 days, 6 days, 7 days, 2 weeks, or more than 2 weeks after the surgery takes place.

15. The method of claim 11, further comprising administering to the subject at least one additional analgesic drug.

16. The method of claim 15, wherein at least one of the following applies:
(a) the amount of the at least one additional analgesic drug administered to the subject along with the composition is lower than the amount of the at least one analgesic drug that needs to be administered to the subject in the absence of the composition to provide a similar level of post-operative pain relief,
(b) the duration of the pain relief provided by the amount of the at least one additional analgesic drug administered to the subject along with the composition is longer than the duration of the pain relief provided by the same amount of at least one additional analgesic drug administered in the absence of the composition.

17. The method of claim 11, wherein the macrophage is allogenic with respect to the subject, or the macrophage is syngenic with respect to the subject.

18. The method of claim 11, wherein the macrophage is obtained from the subject, or obtained from a donated blood.

19. The method of claim 11, wherein the sEV or exosome is administered to the subject parentally.

20. The method of claim 11, wherein the subject is a human.
